(19) United States
(12) Patent Application Publication
(10) Pub. No.: US 2015/0374916 A1
(43) Pub. Date: Dec. 31, 2015

(54) LATERALLY CONNECTED SYRINGES WITH INTEGRATED MIXING HUB AND STATIC MIXER

(71) Applicant: Allergan, Inc., Irvine, CA (US)

(72) Inventors: Tiago Bertolote, Geneve (CH); Ethan Franklin, Goleta, CA (US)

(21) Appl. No.: 14/741,669

(22) Filed: Jun. 17, 2015

Related U.S. Application Data
(60) Provisional application No. 62/018,406, filed on Jun. 27, 2014.

(51) Int. Cl. A61M 5/19; A61M 5/315

(52) U.S. Cl. CPC A61M 5/19; A61M 5/31596; A61M 2005/3123

(57) ABSTRACT

Described herein are laterally connected syringes with an integrated mixing hub and static mixer designed to facilitate mixing two or more substances at specific user-selectable ratios.

### CROSS-REFERENCE

This application claims priority to, and the benefit of, U.S. Provisional Patent Application No. 62/018,406, filed on Jun. 27, 2014, the entire disclosure of which is incorporated herein by this specific reference.

### FIELD

The present invention generally relates to medical injection devices, and more specifically relates to laterally connected syringes with an integrated mixing hub and static mixer configured to facilitate mixing two or more substances at specific user-selectable ratios.

### BACKGROUND

Administering a mixture of materials to a patient usually involves at least two syringes. The two syringes can generally be connected to the ends of one another creating a physically long and burdensome syringe system. Not only is the overall length of the syringe system burdensome, but the practitioner also takes up valuable time in order to prepare and connect the syringes.

Furthermore, connecting at least two syringes with different substances to obtain a mixture does not provide efficient mixing capabilities. For at least these reasons, there remains a need for a syringe assembly which can provide efficient mixing of substances, reduce the overall length of the assembled syringes and reduce the preparation time required to assemble the syringes.

### SUMMARY

Disclosed herein are syringes configured for a lateral connection with an integrated mixing hub and static mixer. The syringes are configured for lateral connection to provide reduced overall length of the syringe assembly and to reduce preparation time, relative to a sequential attachment of two or more conventional syringes. The syringes can be used to mix two or more different substances.

In one embodiment, described herein, are syringe assemblies. A syringe assembly can include: an upper syringe including a lateral inlet; at least one lower syringe including a lateral outlet; and a mixing hub including a static mixer housed in the upper syringe adjacent to the lateral inlet. The syringe assembly can be configured so that the upper syringe is laterally connected to the lower syringe via an attachment between the lateral inlet and the lateral outlet.

In another embodiment, a first substance can be housed in the upper syringe and a second substance can be housed in at least one lower syringe.

Other embodiments include methods of using the syringe assemblies. The methods describe mixing materials in the syringe assembly and extruding the resulting mixture from the syringe assembly. The methods can include: applying a force to a first plunger of at least one lower syringe. The force applied to the first plunger can cause pressure buildup inside the lower syringe translating into a movement of a second plunger housed in the lower syringe adjacent to the lateral outlet. Movement of the second plunger can be configured to cause the lateral outlet to open thereby allowing flow of the second substance into the mixing hub. The first substance and the second substance can mix in the mixing hub via a static mixer to form a mixture. The mixture can be expelled from the upper syringe upon an extrusion force applied to a primary plunger included in the upper syringe.

The lateral outlet can include at least a single section opening. The lateral inlet can include at least two section openings. The single section opening of the lateral outlet can be bigger than the two section openings of the lateral inlet.

The mixing hub can include a static mixer. The static mixer can be comprised of a series of mixing elements. Two or more mixing elements can combine to form a combination of mixing elements.

### BRIEF DESCRIPTION OF THE DRAWINGS

Embodiments of the present description are illustrated by way of example, and not by way of limitation, in the figures of the accompanying drawings and in which like reference numerals refer to similar elements, wherein:

FIG. 1 illustrates a perspective view of a syringe assembly as described herein.

FIG. 2A illustrates a perspective view of a disassembled syringe assembly. FIG. 2B illustrates a perspective view of the syringe assembly in FIG. 2A which has been assembled.

FIG. 3 illustrates a cross-sectional view of the syringe assembly illustrated in FIG. 1.

FIG. 4 illustrates a cross-sectional view of a lateral outlet and a lateral inlet of an assembled syringe assembly.

FIGS. 5A-B illustrate a cross-sectional view of the fluid separation within the syringe assembly. FIG. 5A illustrates a loaded syringe assembly system. FIG. 5B illustrates movement of a second plunger opening the lateral outlet and allowing the second substance to flow from a lateral outlet to a lateral inlet and into a mixing hub.

FIGS. 6A-D illustrate different mixing elements which make up the static mixer.

FIGS. 7A-C illustrate various combinations of mixing elements illustrated in FIGS. 6A-D.

### DETAILED DESCRIPTION

Generally described herein are syringe assemblies comprised of syringes configured for a lateral connection and include an integrated mixing hub and static mixer. The syringes are configured for lateral connection to provide reduced overall length of the assembly and to reduce preparation time. In other embodiments, a syringe assembly as described herein can be supplied already connected as a single article. In still other embodiments, a syringe assembly as described herein can be supplied separately and connected by the physician via a snap fit, a friction fit, a lock fit, a glue, an adhesive or a combination thereof.

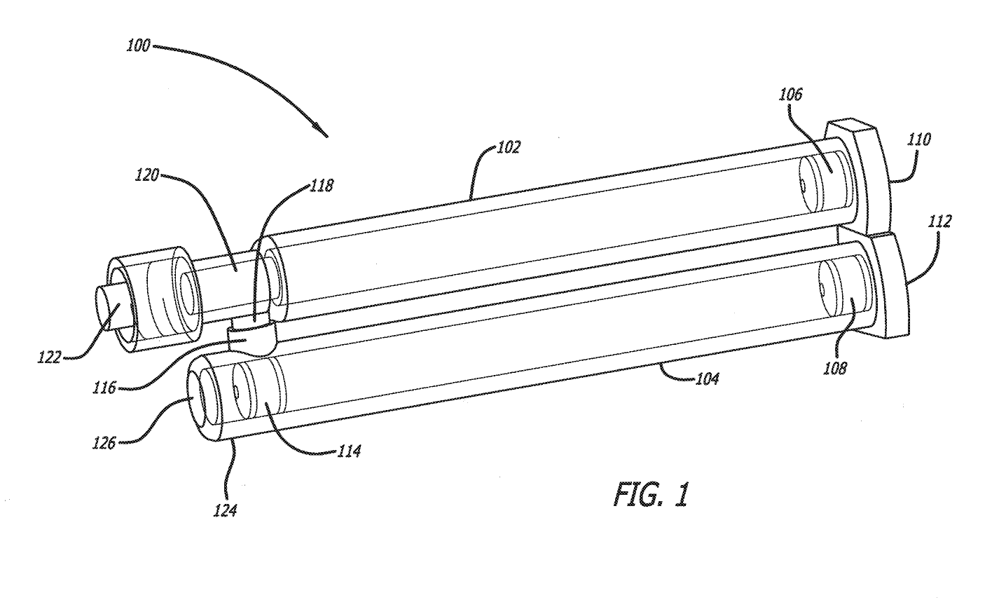

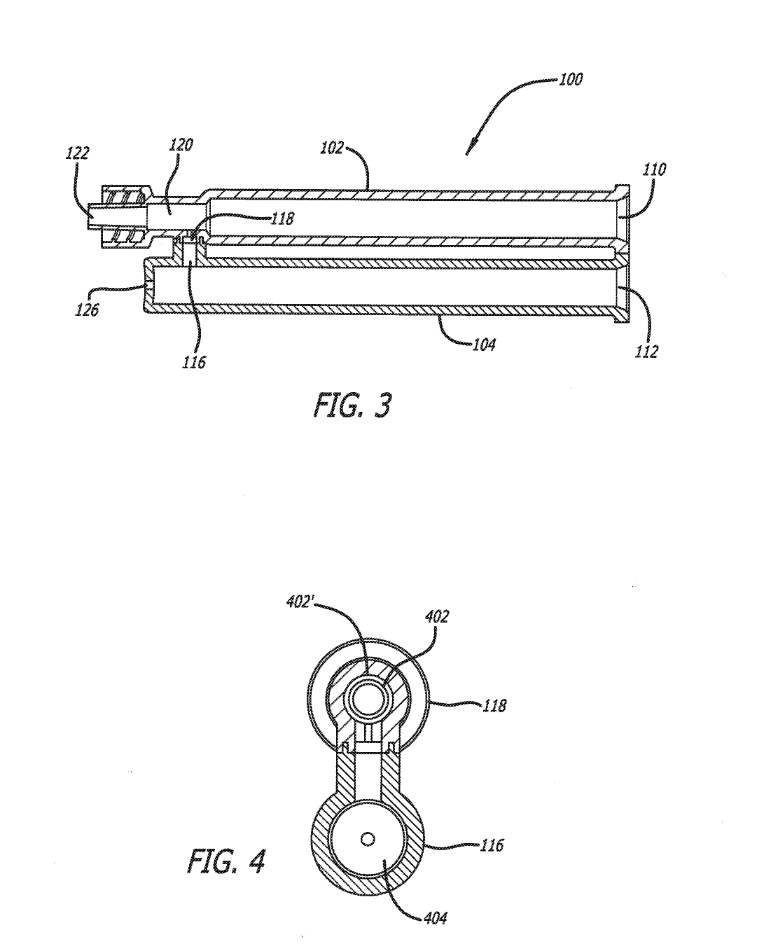

As illustrated in FIG. 1, syringe assembly 100 can be supplied connected as a single article. Syringe assembly 100 can generally include upper syringe 102, lower syringe 104, primary plunger 106, first plunger 108, second plunger 114, lateral outlet 116, lateral inlet 118 and mixing hub 120. A cross-sectional view of syringe assembly 100 is illustrated in FIG. 3.

Upper syringe 102 and lower syringe 104 can be disposable. In some embodiments, upper syringe 102 and lower syringe 104 can be disposable after a single use.

Upper syringe 102 includes primary plunger 106, mixing hub 120, plunger head 110, attachment portion 122, and lateral inlet 118. Lower syringe 104 includes first plunger 108, plunger head 112, second plunger 114 and lateral outlet 116. Lateral inlet 118 of first syringe 102 is configured to fit/attach to lateral outlet 116 of lower syringe 104. Lateral inlet 118 can fit into/attach to lateral outlet 116 via a snap-fit, a friction fit, a lock fit, a glue, an adhesive or a combination thereof.

Upper syringe 102 can house a first substance and lower syringe 104 can house a second substance. The first substance and the second substance can be different. The first substance and the second substance can mix in mixing hub 120 to form a mixture which can be extruded out of upper syringe 102.

In some embodiments, the first substance can be a gel and/or a fluid. For example the first substance can be a dermal filler such as, but not limited to, Juvederm® Voluma XC, manufactured by Allergan, Inc. In some embodiments, the second substance can be a gel and/or a fluid. For example the second substance can be a dilutant such as, but not limited to, saline or PBS.

In yet another embodiment, the first substance can be a dilutant and the second substance can be a dermal filler.

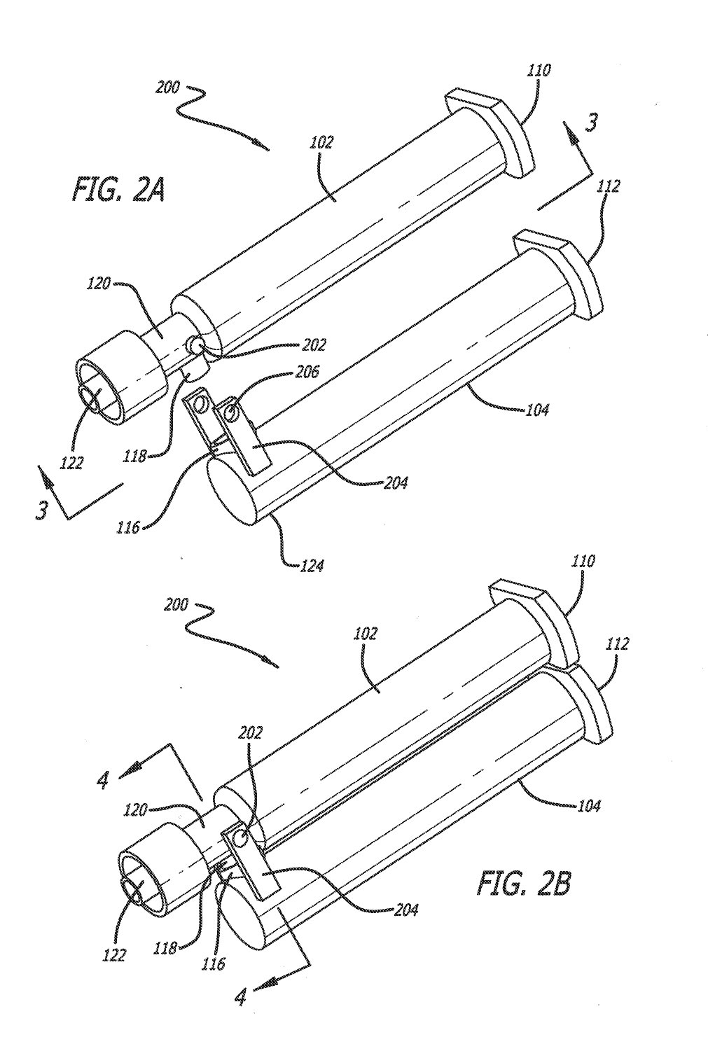

Upper syringe 102 and lower syringe 104 can be supplied separately and connected by a physician prior to use as illustrated in FIGS. 2A-B. In one embodiment, when upper syringe 102 and lower syringe 104 are supplied separately, upper syringe 102 can include a seal or pierceable membrane located on the underside of lateral inlet 118 to avoid the first substance coming into contact with the external atmosphere.

FIG. 2A illustrates syringe assembly 200 supplied unconnected and FIG. 2B illustrates syringe assembly 200 connected upon being supplied separately. Attachment hinge 204 can be near proximal end 124 of lower syringe 104. Attachment hinge 204 can include two or more openings 206 configured to attach to attachment knobs 202 which can be located on opposite sides of mixing hub 120 (only one attachment knob is shown in FIGS. 2A-B). Attachment hinge 204 can fit on or over attachment knobs 202 securing upper syringe 102 and lower syringe 104 in a connected lateral configuration as exemplified in FIG. 2B. Attachment hinge 204 can engage with attachment knobs 202 using a friction fit, a locking fit where attachment hinge 204 includes locking features that snap and lock once the two parts are engaged, or a glue engagement wherein attachment hinge 204 and attachment knobs 202 are glued together.

Lateral outlet 116 can comprise at least a single section opening 404. Lateral inlet 118 can comprise at least two section openings 402 and 402′. Single section 404 can be bigger than section openings 402 and 402′ as illustrated in FIG. 4. This configuration can improve mixing of the first substance and the second substance. For example, in one embodiment, syringe assembly 100 includes the lateral inlet and lateral outlet configuration as illustrated in FIG. 4. The first substance can be a dermal filler and the second substance can be a dilutant. This configuration improves mixing by decreasing the dilutant's flow section, which causes the flow velocity to increase, and thus the dilutant “jets” into the dermal filler present in the mixing hub. This configuration can further create turbulences that improve mixing.

Yet another advantage of syringe assembly 100 is that when it is pre-assembled the first substance and the second substance are separated from each other until the mixing and injection process takes place. This is advantageous as that any undesirable effect of a premature mixing is excluded. The separation of substances can be created by means of second plunger 114 housed in lower syringe 104. Second plunger 114 can be positioned during manufacture so that it obturates lateral outlet 116. Second plunger 114 not only prevents the second substance from mixing with the first substance but it also prevents the first substance from being in contact with the external atmosphere.

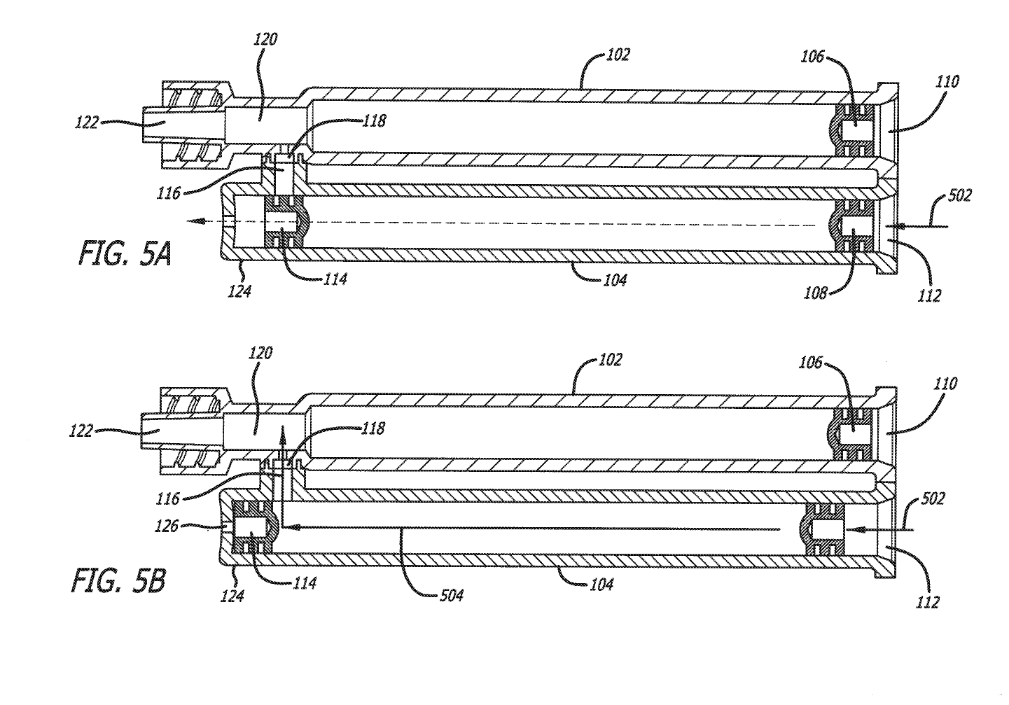

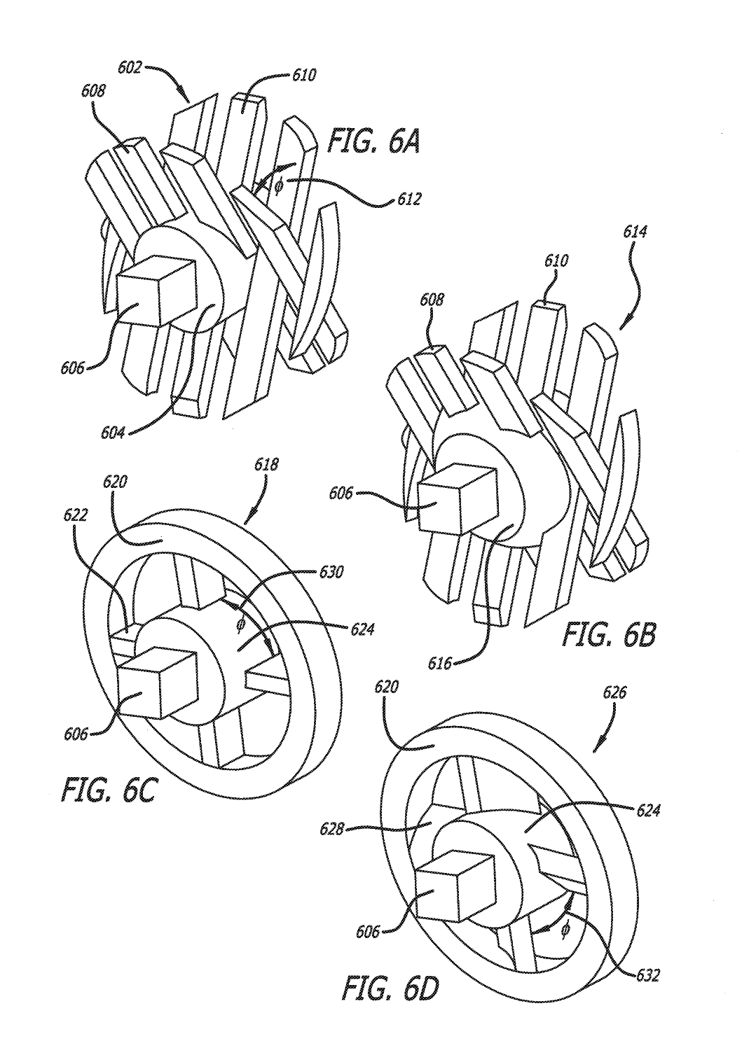

FIGS. 5A-B illustrate a cross-sectional view of syringe assembly 100 when it is pre-assembled. FIG. 5A illustrates syringe assembly 100 prior to use. Second plunger 114 is intact and obtrurates lateral outlet 116. FIG. 5B illustrates syringe assembly 100 in use when second plunger 114 has been moved opening lateral outlet 116. Opening lateral outlet 116 allows the second substance to take flow path 504, flowing towards proximal end 124 of lower syringe 104 and up through lateral outlet 116 and lateral inlet 118 into mixing hub 120 where the first substance is present and mixing can begin.

Second plunger 114 can be moved by applying force to plunger head 112 of lower syringe 104. Since lateral outlet 116 is closed prior to use applying force to plunger head 112 can cause a pressure buildup in lower syringe 104 which translates into movement of second plunger 114. Second plunger 114 moves towards proximal end 124 of lower syringe 104 (as illustrated in FIG. 5B) creating a path between the two syringes by fully opening lateral outlet 116. Once lateral outlet 116 is open the second substance can start flowing along flow path 504 towards proximal end 124 of lower syringe 104 flowing up through lateral outlet 116 to lateral inlet 118 and into mixing hub 120. Lower syringe 104 can further comprise venting hole 126 near proximal end 124. Venting hole 126 can ensure that no air gets trapped by second plunger 114.

The force applied to plunger head 112 of lower syringe 104 can be can be greater than about 1 Newton (N), greater than about 2 N, greater than about 3 N, greater than about 4 N, greater than about 5 N, greater than about 10 N, greater than about 20 N, greater than about 40 N, greater than about 60 N, greater than about 80 N, greater than about 100 N, between about 1 N and about 20 N, between about 1 N and about 100 N, or between about 20 N and about 100 N.

Mixing hub 120 can include a static mixer (not shown). The static mixer can be comprised of a series of mixing elements. The mixing elements can be different and serve different purposes, so that once combined they provide efficient mixing. FIGS. 6A-D show different types of mixing elements that can be used with the assemblies described herein.

FIG. 6A depicts first mixing element 602 which can include wheel 604, first blade 608, second blade 610 and knob 606. First blade 608 and second blade 610 can be attached to wheel 604 and can have predetermined distance 612 between them. First blade 608 and second blade 610 can be attached to wheel 604 via any attachment mechanism including, but not limited to, a glue or an adhesive or a combination thereof. Predetermined distance 612 can be about 15 degrees, about 20 degrees, about 25 degrees, about 30 degrees, about 35 degrees, about 40 degrees, about 45 degrees, about 50 degrees, about 55 degrees, about 60 degrees, about 65 degrees, about 70 degrees, about 75 degrees, about 80 degrees, about 85 degrees, about 90 degrees, between about 10 degrees to about 30 degrees, between about 20 degrees to about 40 degrees, between about 30 degrees to about 50 degrees, between about 40 degrees to about 70 degrees, between about 50 degrees to about 80 degrees, between about 60 degrees to 90 about degrees, less than 15 about degrees, less than about 25 degrees, less than about 35 degrees, less than about 45 degrees, less than about 55 degrees, less than about 65 degrees, less than about 75 degrees, less than about 80 degrees, less than about 95 degrees, more than about 10 degrees, more than about 20 degrees, more than about 30 degrees, more than about 40 degrees, more than about 50 degrees, more than about 60 degrees, more than about 70 degrees, more than about 80 degrees, more than about 90 degrees.

First blade 608 and second blade 610 can be any length. For example, first blade 608 and second blade 610 can be about 0.2 cm, about 0.4 cm, about 0.6 cm, about 0.8 cm, about 1.2 cm, about 1.4 cm, about 1.6 cm, about 1.8 cm, about 2.0 cm, about 2.2 cm, about 2.4 cm, about 2.6 cm, about 2.8 cm, about 3.0 cm, about 4.0 cm, about 5.0 cm, about 6.0 cm, about 7.0 cm, about 8.0 cm in length. In another embodiment, first blade 608 and second blade 610 can be between about 0.1 cm to about 2.0 cm, between about 1.0 cm to about 2.0 cm, between about 2.0 cm to about 3.0 cm, between about 3.0 cm to about 4.0 cm, between about 4.0 cm to about 5.0 cm, between about 5.0 cm to about 6.0 cm, between about 6.0 cm to about 7.0 cm, between about 7.0 cm to about 8.0 cm, less than about 0.1 cm, less than about 0.2 cm, less than about 0.3 cm, less than about 0.4 cm, less than about 0.5 cm, less than about 0.6 cm, less than about 0.7 cm, less than about 1.0 cm, less than about 2.0 cm, less than about 3.0 cm, less than about 4.0 cm, less than about 5.0 cm, less than about 6.0 cm, less than about 7.0 cm, less than about 8.0 cm, more than about 0.1 cm, more than about 0.2 cm, more than about 0.3 cm, more than about 0.4 cm, more than about 0.5 cm, more than about 0.6 cm, more than about 0.7 cm, more than about 1.0 cm, more than about 2.0 cm, more than about 3.0 cm, more than about 4.0 cm, more than about 5.0 cm, more than about 6.0 cm, more than about 7.0 cm, more than about 8.0 cm in length.

Wheel 604 can be about 1.0 cm, about 1.2 cm, about 1.4 cm, about 1.6 cm, about 1.8 cm, about 2.0 cm, about 2.2 cm, about 2.4 cm, about 2.6 cm, about 2.8 cm, about 3.0 cm, about 4.0 cm or about 5.0 cm in diameter. In another embodiment, wheel 604 can be between about 1.0 cm to about 2.0 cm, between about 2.0 cm to about 3.0 cm, between about 3.0 cm to about 4.0 cm, between about 4.0 cm to about 5.0 cm, less than about 1.0 cm, less than about 2.0 cm, less than about 3.0 cm, less than about 4.0 cm, less than about 5.0 cm, more than about 1.0 cm, more than about 2.0 cm, more than about 3.0 cm, more than about 4.0 cm or more than about 5.0 cm in diameter.

Knob 606 can be attached to about the center, about 0.01 cm from the center, about 0.02 cm, about 0.03 cm from the center, about 0.04 cm from the center, about 0.05 cm from the center, less than about 0.01 cm from the center, less than about 0.02 cm, less than about 0.03 cm from the center, less than about 0.04 cm from the center, less than about 0.05 cm from the center, more than about 0.01 cm from the center, more than about 0.02 cm, more than about 0.03 cm from the center, more than about 0.04 cm from the center or more than about 0.05 cm from the center of wheel 604. Knob 606 can be attached to wheel 604 via any attachment mechanism including, but not limited to, a glue or an adhesive or a combination thereof. Wheel 604 can be any shape and generally can be substantially cylindrical. Wheel 604 can include about 1, 2, 3, 4, 5, 6, 7, 8, 9, 10, 11, 12, 13, 14 or more blades. Knob 606 can be any shape and generally can be substantially square. First mixing element 602 can fragment the flow and create velocity gradients, while pushing the substances and/or fluids toward the walls of the mixing hub.

FIG. 6B depicts second mixing element 614 which utilizes the same mechanism as first mixing element 602. Second mixing element 614 can include first blade 608, second blade 610, knob 606, and center hub 616. Second mixing element 614 can first blade 608 and second blade 610 which are shorter than first blade 608 and second blade 610 in comparison to first mixing element 602. Center hub 616 can have a wider portion or diameter than wheel 604. Center hub 616 generally has a wider central portion than wheel 604, thus occupying a larger portion of the mixing hub.

Center hub 616 can be about be about 1.0 cm, about 1.2 cm, about 1.4 cm, about 1.6 cm, about 1.8 cm, about 2.0 cm, about 2.2 cm, about 2.4 cm, about 2.6 cm, about 2.8 cm, about 3.0 cm, about 3.5 cm, about 4.0 cm, about 4.5. cm, about 5.0 cm, about 5.5 cm, about 6.0 cm, about 6.5 cm, about 7.0 cm, about 7.5 cm or about 8.0 in diameter. In another embodiment, center hub 616 can be between about 1.0 cm to about 2.0 cm, between about 2.0 cm to about 3.0 cm, between about 3.0 cm to about 4.0 cm, between about 4.0 cm to about 5.0 cm, between about 5.0 cm to about 6.0 cm, between about 6.0 cm to about 7.0 cm, between about 7.0 cm to about 8.0 cm, less than about 1.0 cm, less than about 2.0 cm, less than about 3.0 cm, less than about 4.0 cm, less than about 5.0 cm, less than about 6.0 cm, less than about 7.0 cm, less than about 8.0 cm, more than about 1.0 cm, more than about 2.0 cm, more than about 3.0 cm, more than about 4.0 cm, more than about 5.0 cm, more than about 6.0 cm, more than about 7.0 cm or more than about 8.0 cm in diameter.

First blade 608 and second blade 610 are attached to center hub 616 and can have predetermined distance 612 between them. First blade 608 and second blade 610 can be attached to center hub 616 via any attachment mechanism including, but not limited to, a glue or an adhesive or a combination thereof.

Knob 606 can be attached to center hub 616 via any attachment mechanism including, but not limited to, a glue or an adhesive or a combination thereof. Center hub 616 can be any shape and generally can be substantially cylindrical. Center hub 616 can include about 1, 2, 3, 4, 5, 6, 7, 8, 9, 10, 11, 12, 13, 14 or more blades. Knob 606 can be any shape and generally can be substantially square.

FIG. 6C depicts third mixing element 618. Third mixing element 618 can concentrate a flow closer to the center of a mixing hub. Third mixing element 618 can include spoke(s) 622, inner wheel 624, outer wheel 620 and knob 606. Spoke(s) 622 connects inner wheel 624 to outer wheel 620. Spoke(s) 622 can be attached to inner wheel 624 and outer wheel 620 via any attachment mechanism including, but not limited to, a glue or an adhesive or a combination thereof. Third mixing element 618 can include 1, 2, 3, 4, 5, 6, 7, 8, 9, 10, 11, 12 or more spoke(s) 622 but generally includes at least four spoke(s) 622. Knob 606 can be attached to inner wheel 624 via any attachment mechanism including, but not limited to, a glue or an adhesive or a combination thereof. Inner wheel 624 and outer wheel 620 can be any shape and generally can be substantially cylindrical.

Spoke(s) 622 can be any length. For example, spoke(s) 622 can be about 0.2 cm, about 0.4 cm, about 0.6 cm, about 0.8 cm, about 1.2 cm, about 1.4 cm, about 1.6 cm, about 1.8 cm, about 2.0 cm, about 2.2 cm, about 2.4 cm, about 2.6 cm, about 2.8 cm, about 3.0 cm, about 4.0 cm, about 5.0 cm, about 6.0 cm, about 7.0 cm, about 8.0 cm in length. In another embodiment, spoke(s) 622 can be between about 0.1 cm to about 2.0 cm, between about 1.0 cm to about 2.0 cm, between about 2.0 cm to about 3.0 cm, between about 3.0 cm to about 4.0 cm, between about 4.0 cm to about 5.0 cm, between about 5.0 cm to about 6.0 cm, between about 6.0 cm to about 7.0 cm, between about 7.0 cm to about 8.0 cm, less than about 0.1 cm, less than about 0.2 cm, less than about 0.3 cm, less than about 0.4 cm, less than about 0.5 cm, less than about 0.6 cm, less than about 0.7 cm, less than about 1.0 cm, less than about 2.0 cm, less than about 3.0 cm, less than about 4.0 cm, less than about 5.0 cm, less than about 6.0 cm, less than about 7.0 cm, less than about 8.0 cm, more than about 0.1 cm, more than about 0.2 cm, more than about 0.3 cm, more than about 0.4 cm, more than about 0.5 cm, more than about 0.6 cm, more than about 0.7 cm, more than about 1.0 cm, more than about 2.0 cm, more than about 3.0 cm, more than about 4.0 cm, more than about 5.0 cm, more than about 6.0 cm, more than about 7.0 cm, more than about 8.0 cm in length.

Inner wheel 624 can be about 1.0 cm, about 1.2 cm, about 1.4 cm, about 1.6 cm, about 1.8 cm, about 2.0 cm, about 2.2 cm, about 2.4 cm, about 2.6 cm, about 2.8 cm, about 3.0 cm, about 4.0 cm or about 5.0 cm in diameter. In another embodiment, inner wheel 624 can be between about 1.0 cm to about 2.0 cm, between about 2.0 cm to about 3.0 cm, between about 3.0 cm to about 4.0 cm, between about 4.0 cm to about 5.0 cm, less than about 1.0 cm, less than about 2.0 cm, less than about 3.0 cm, less than about 4.0 cm, less than about 5.0 cm, more than about 1.0 cm, more than about 2.0 cm, more than about 3.0 cm, more than about 4.0 cm or more than about 5.0 cm in diameter.

Outer wheel 620 can be about 1.0 cm, about 1.2 cm, about 1.4 cm, about 1.6 cm, about 1.8 cm, about 2.0 cm, about 2.2 cm, about 2.4 cm, about 2.6 cm, about 2.8 cm, about 3.0 cm, about 4.0 cm, about 5.0 cm, about 6.0 cm, about 7.0 cm, about 8.0 cm, about 9.0 cm, about 10.0 cm, about 11.0 cm, about 12.0 cm, about 13.0 cm, about 14.0 cm, about 15.0 cm or about 16.0 cm in diameter. In another embodiment, outer wheel 620 can be between about 1.0 cm to about 2.0 cm, between about 2.0 cm to about 3.0 cm, between about 3.0 cm to about 4.0 cm, between about 4.0 cm to about 5.0 cm, between about 5.0 cm to about 6.0 cm, between about 6.0 cm to about 7.0 cm, between about 7.0 cm to about 8.0 cm, between about 8.0 cm to about 9.0 cm, between about 9.0 cm to about 10.0 cm, between about 10.0 cm to about 11.0 cm, between about 11.0 cm to about 12.0 cm, between about 12.0 cm to about 13.0 cm, between about 13.0 cm to about 14.0 cm, between about 14.0 cm to about 15.0 cm, between about 15.0 cm to about 16.0 cm, less than about 1.0 cm, less than about 2.0 cm, less than about 3.0 cm, less than about 4.0 cm, less than about 5.0 cm, less than about 6.0 cm, less than about 7.0 cm, less than about 8.0 cm, less than about 9.0 cm, less than about 10.0 cm, less than about 11.0 cm, less than about 12.0 cm, less than about 13.0 cm, less than about 14.0 cm, less than about 15.0 cm, less than about 16.0 cm, more than about 1.0 cm, more than about 2.0 cm, more than about 3.0 cm, more than about 4.0 cm, more than about 5.0 cm, more than about 6.0 cm, more than about 7.0 cm, more than about 8.0 cm, more than about 9.0 cm, more than about 10.0 cm, more than about 11.0 cm, more than about 12.0 cm, more than about 13.0 cm, more than about 14.0 cm, more than about 15.0 cm or more than about 16.0 cm in diameter.

In one embodiment, third mixing element 618 can have four spoke(s) 622 as illustrated in FIG. 6C which can be separated by angle 630. Generally angle 630 can be about 90 degrees. In other embodiments angle 630 can be about 10 degrees, about 15 degrees, about 20 degrees, about 25 degrees, about 30 degrees, about 35 degrees, about 40 degrees, about 45 degrees, about 50 degrees, about 55 degrees, about 60 degrees, about 65 degrees, about 70 degrees, about 75 degrees, about 80 degrees, about 85 degrees, about 90 degrees, between about 10 degrees to about 20 degrees, between about 15 degrees to about 25 degrees, between about 20 degrees to about 30 degrees, between about 25 degrees to about 35 degrees, between about 30 degrees to about 40 degrees, between about 35 degrees to about 45 degrees, between about 40 degrees to about 50 degrees, between about 55 degrees to about 65 degrees, between about 60 degrees to about 70 degrees, between about 65 degrees to about 75 degrees, between about 70 degrees to about 80 degrees, between about 75 degrees to about 85 degrees, between about 80 degrees to about 90 degrees, between about 85 degrees to about 95 degrees or between about 90 degrees to 100 degrees.

In yet another embodiment, angle 630 can be less than about 10 degrees, less than about 15 degrees, less than about 20 degrees, less than about 25 degrees, less than about 30 degrees, less than about 35 degrees, less than about 40 degrees, less than about 45 degrees, less than about 50 degrees, less than about 55 degrees, less than about 60 degrees, less than about 65 degrees, less than about 70 degrees, less than about 75 degrees, less than about 80 degrees, less than about 85 degrees, less than about 90 degrees, more than about 10 degrees, more than about 15 degrees, more than about 20 degrees, more than about 25 degrees, more than about 30 degrees, more than about 35 degrees, more than about 40 degrees, more than about 45 degrees, more than about 50 degrees, more than about 55 degrees, more than about 60 degrees, more than about 65 degrees, more than about 70 degrees, more than about 75 degrees, more than about 80 degrees, more than about 85 degrees, more than about 90 degrees.

Fourth mixing element 626 illustrated in FIG. 6D is similar to third mixing element 618 but with angled spoke(s) 628 in order to generate a rotation of the flow. Fourth mixing element 626 can include angled spoke(s) 628, inner wheel 624, outer wheel 620 and knob 606. Angled spoke(s) 628 connect inner wheel 624 to outer wheel 620. Angled spoke(s) 628 can be attached to inner wheel 624 and outer wheel 620 via any attachment mechanism including, but not limited to, a glue or an adhesive or a combination thereof. Fourth mixing element 626 can include 1, 2, 3, 4, 5, 6, 7, 8, 9, 10, 11, 12 or more angled spoke(s) 628 but generally includes at least four angled spoke(s) 628. Knob 606 can be attached to inner wheel 624 via any attachment mechanism including, but not limited to, a glue or an adhesive or a combination thereof. Inner wheel 624 and outer wheel 620 can be any shape and generally can be substantially cylindrical.

Angled spoke(s) 628 can be any length. For example, angled spoke(s) 628 can be about 0.2 cm, about 0.4 cm, about 0.6 cm, about 0.8 cm, about 1.2 cm, about 1.4 cm, about 1.6 cm, about 1.8 cm, about 2.0 cm, about 2.2 cm, about 2.4 cm, about 2.6 cm, about 2.8 cm, about 3.0 cm, about 4.0 cm, about 5.0 cm, about 6.0 cm, about 7.0 cm, about 8.0 cm in length. In another embodiment, angled spoke(s) 628 can be between about 0.1 cm to about 2.0 cm, between about 1.0 cm to about 2.0 cm, between about 2.0 cm to about 3.0 cm, between about 3.0 cm to about 4.0 cm, between about 4.0 cm to about 5.0 cm, between about 5.0 cm to about 6.0 cm, between about 6.0 cm to about 7.0 cm, between about 7.0 cm to about 8.0 cm, less than about 0.1 cm, less than about 0.2 cm, less than about 0.3 cm, less than about 0.4 cm, less than about 0.5 cm, less than about 0.6 cm, less than about 0.7 cm, less than about 1.0 cm, less than about 2.0 cm, less than about 3.0 cm, less than about 4.0 cm, less than about 5.0 cm, less than about 6.0 cm, less than about 7.0 cm, less than about 8.0 cm, more than about 0.1 cm, more than about 0.2 cm, more than about 0.3 cm, more than about 0.4 cm, more than about 0.5 cm, more than about 0.6 cm, more than about 0.7 cm, more than about 1.0 cm, more than about 2.0 cm, more than about 3.0 cm, more than about 4.0 cm, more than about 5.0 cm, more than about 6.0 cm, more than about 7.0 cm, more than about 8.0 cm in length.

In one embodiment, fourth mixing element 626 can have four angled spoke(s) 628 as illustrated in FIG. 6D which can be separated by second angle 632. Generally second angle 632 can be about 90 degrees. In other embodiments second angle 632 can be about 10 degrees, about 15 degrees, about 20 degrees, about 25 degrees, about 30 degrees, about 35 degrees, about 40 degrees, about 45 degrees, about 50 degrees, about 55 degrees, about 60 degrees, about 65 degrees, about 70 degrees, about 75 degrees, about 80 degrees, about 85 degrees, about 90 degrees, between about 10 degrees to about 20 degrees, between about 15 degrees to about 25 degrees, between about 20 degrees to about 30 degrees, between about 25 degrees to about 35 degrees, between about 30 degrees to about 40 degrees, between about 35 degrees to about 45 degrees, between about 40 degrees to about 50 degrees, between about 55 degrees to about 65 degrees, between about 60 degrees to about 70 degrees, between about 65 degrees to about 75 degrees, between about 70 degrees to about 80 degrees, between about 75 degrees to about 85 degrees, between about 80 degrees to about 90 degrees, between about 85 degrees to about 95 degrees or between about 90 degrees to 100 degrees.

In yet another embodiment, second angle 632 can be less than about 10 degrees, less than about 15 degrees, less than about 20 degrees, less than about 25 degrees, less than about 30 degrees, less than about 35 degrees, less than about 40 degrees, less than about 45 degrees, less than about 50 degrees, less than about 55 degrees, less than about 60 degrees, less than about 65 degrees, less than about 70 degrees, less than about 75 degrees, less than about 80 degrees, less than about 85 degrees, less than about 90 degrees, more than about 10 degrees, more than about 15 degrees, more than about 20 degrees, more than about 25 degrees, more than about 30 degrees, more than about 35 degrees, more than about 40 degrees, more than about 45 degrees, more than about 50 degrees, more than about 55 degrees, more than about 60 degrees, more than about 65 degrees, more than about 70 degrees, more than about 75 degrees, more than about 80 degrees, more than about 85 degrees, more than about 90 degrees.

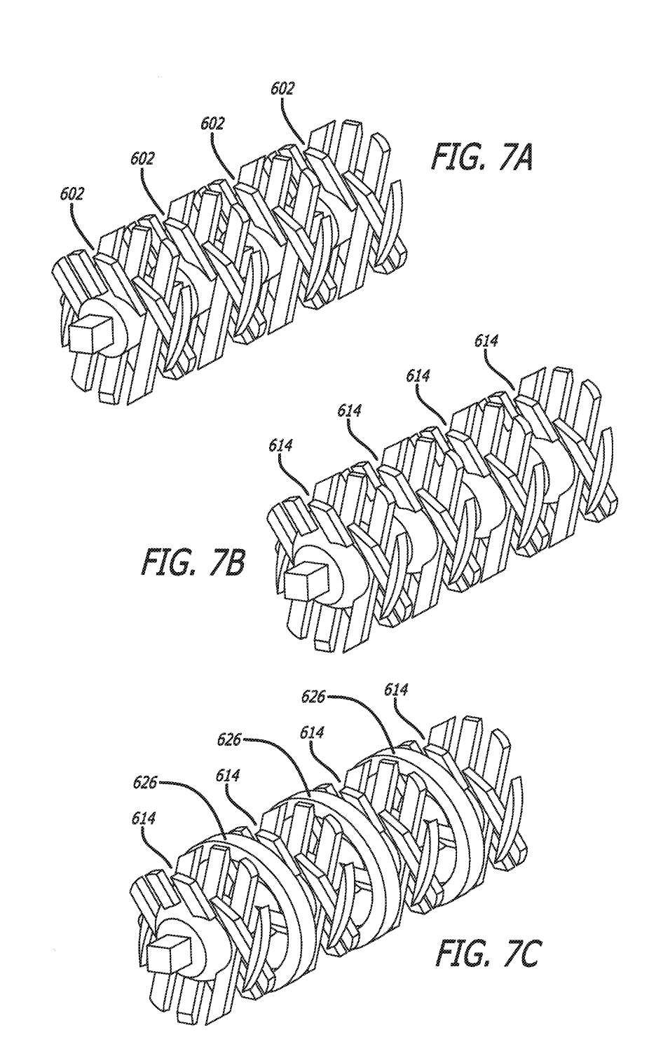

First mixing element 602, second mixing element 614, third mixing element 618 and fourth mixing element 626 can be combined to achieve different mixing results. Some examples of such combinations are depicted in FIGS. 7A-C and are solely for illustration purposes and not considered to be limiting. FIG. 7A illustrates a combination of four first mixing element(s) 602 connected with a 90° rotation. FIG. 7B illustrates a combination of four second mixing element(s) 614. FIG. 7C illustrates the combination of four second mixing element(s) 614 with three fourth mixing element(s) 626 in an alternating pattern which can be reversed.

Once the mixing has occurred and a mixture with a ratio selected by the user has been achieved the mixture can be extruded from syringe assembly 100. The selected ratio can be any ratio desired by the user. For example, selected ratios can include, but are not limited to, about 15% of the first substance and about 85% of the second substance, about 25% of the first substance and about 75% of the second substance, about 35% of the first substance and about 65% of the second substance, about 45% of the first substance and about 55% of the second substance, about 65% of the first substance and about 35% of the second substance, about 75% of the first substance and about 25% of the second substance, about 85% of the first substance and about 15% of the second substance, about 55% of the first substance and about 45% of the second substance, about 0% of the first substance and about 100% of the second substance, about 10% of the first substance and about 90% of the second substance, about 20% of the first substance and about 80% of the second substance, about 30% of the first substance and about 70% of the second substance, about 40% of the first substance and about 60% of the second substance, about 50% of the first substance and about 50% of the second substance, about 60% of the first substance and about 40% of the second substance, about 70% of the first substance and about 30% of the second substance, about 80% of the first substance and about 20% of the second substance, about 90% of the first substance and about 10% of the second substance, about 100% of the first substance and about 0% of the second substance, between about 0-10% of the first substance and 90-100% of the second substance, between about 10-20% of the first substance and 80-90% of the second substance, between about 20-30% of the first substance and 70-80% of the second substance, between about 30-40% of the first substance and 60-70% of the second substance, between about 40-50% of the first substance and 50-60% of the second substance, between about 60-70% of the first substance and 30-40% of the second substance, between about 70-80% of the first substance and 20-30% of the second substance, between about 80-90% of the first substance and 10-20% of the second substance or between about 90-100% of the first substance and 0-10% of the second substance

An extrusion force applied to primary plunger 110 of upper syringe 102 can cause the mixture to extrude through attachment portion 122 and out, for example, but not limited to, a cannula or needle (needle and/or cannula not shown in the Figures) which is configured to attach to attachment portion 122.

The extrusion force applied to primary plunger 110 can be greater than about 1 Newton (N), greater than about 2 N, greater than about 3 N, greater than about 4 N, greater than about 5 N, greater than about 10 N, greater than about 20 N, greater than about 40 N, greater than about 60 N, greater than about 80 N, greater than about 100 N, between about 1 N and about 20 N, between about 1 N and about 100 N, or between about 20 N and about 100 N.

A cannula or a needle as used herein can be a 10, 12, 14, 16, 18, 20, 22, 25, 27, 30 up to 33 gauge, or other gauges. In one embodiment, the needle is a 30 gauge. In some embodiments, the needle gauge may be one suitable for fat grafting or dermal filler purposes. The length of a needle can be any appropriate length known in the art. In one embodiment, the needle length is about 1/16 inch to about 3 inches, about 1/16 inch to about 2 inches. In another embodiment, the needle length is 1.5 inches. A cannula or a needle may be blunt or sharp tipped.

The syringe assembly described herein can allow an operator to easily mix substances at specific user-selectable ratios and then extrude the resulting mixture through any needle known to one skilled in the art. In one embodiment, the mixture is extruded through a high gauge needle, for example a 30 gauge, 1.5 inch needle. The syringe assembly can be supplied connected or unconnected. Either way the mixing hub and the lateral connection of the syringes reduces the overall length of the syringe assembly. Furthermore, by supplying the syringe system pre-connected a physician's preparation time is reduced. In addition, the syringes can be disposable which allows for easy clean-up and prevents cross-contamination between patients.

Kits including a syringe assembly as described herein are also contemplated. A kit can include an upper syringe with a first substance and a lower syringe with a second substance configured to connect via a lateral inlet and lateral outlet and instructions for use. In other embodiments, a kit can include a pre-connected syringe assembly and instructions for use.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A syringe assembly comprising:
an upper syringe including a lateral inlet;
a lower syringe including a lateral outlet;
a mixing hub including a static mixer housed in the upper syringe adjacent to the lateral inlet; and
wherein the upper syringe is configured to be laterally connected to the lower syringe via an attachment between the lateral inlet and the lateral outlet.

2. The syringe assembly of claim 1, further comprising a second plunger housed in the lower syringe blocking the lateral outlet.

3. The syringe assembly of claim 2, wherein the second plunger is positioned to obturate the lateral outlet prior to use.

4. The syringe assembly of claim 2, wherein a force applied to a first plunger of the lower syringe causes pressure buildup inside the lower syringe translating into a movement of the second plunger towards proximal end of lower syringe thereby opening the lateral outlet.

5. The syringe assembly of claim 4, wherein opening of the lateral outlet allows flow of a second substance towards the proximal end of the lower syringe and into the lateral outlet and the lateral inlet into the mixing hub.

6. The syringe assembly of claim 5, wherein the second substance flows into the mixing hub where a first substance is present.

7. The syringe assembly of claim 6, wherein the mixing hub includes a static mixer to facilitate the mixing of the first substance and the second substance to form a mixture.

8. The syringe assembly of claim 7, wherein the static mixer comprises a series of mixing elements.

9. The syringe assembly of claim 8, wherein the mixing elements are selected from a group consisting of a first mixing element, a second mixing element, a third mixing element, a fourth mixing element, or a combination thereof.

10. The syringe assembly of claim 9, wherein the mixing elements are a combination of four second mixing elements and three fourth mixing elements.

11. The syringe assembly of claim 7, further comprising an attachment portion at proximal end of the upper syringe configured to attach a needle wherein the mixture is extruded by an extrusion force applied to a primary plunger housed in the upper syringe causing the mixture in the mixing hub to be driven through the attachment portion and out the needle.

12. The syringe assembly of claim 11, wherein the needle is a 30 gauge 1.5 inch needle.

13. The syringe assembly of claim 1, wherein the lateral inlet comprises at least two section openings.

14. The syringe assembly of claim 1, wherein the lateral outlet comprises at least a single section opening.

15. The syringe assembly of claim 6, wherein the first substance is a dermal filler.

16. The syringe assembly of claim 5, wherein the second substance is a dilutant.

17. The syringe assembly of claim 1, further comprising a venting hole at proximal end of the lower syringe.

18. The syringe assembly of claim 1, wherein the attachment of the lateral outlet and lateral inlet is attached by a snap fit, a friction fit, a lock fit, an adhesive, a glue, a hinge or a combination thereof.
